(19) United States
(12) Patent Application Publication
(10) Pub. No.: US 2026/0345614 A1
(43) Pub. Date: Nov. 5, 2026

(54) RESTLESS LEG SYNDROME (RLS) AND PERIODIC LEG MOVEMENTS (PLMS) DURING SLEEP: BIOMARKER AND DRUG TARGET

(71) Applicant: The Board of Trustees of the Leland Stanford Junior University, Stanford, CA (US)

(72) Inventors: Emmanuel Mignot, Redwood City, CA (US); Katie Cederberg, Redwood City, CA (US)

(21) Appl. No.: 19/486,345

(22) PCT Filed: May 30, 2024
(86) PCT No.: PCT/US2024/031625

§ 371 Date: Nov. 20, 2025

Related U.S. Application Data
(60) Provisional application No. 63/469,620, filed on May 30, 2023.

(51) Int. Cl. A61K 31/519; A61K 31/381; A61K 31/4045; A61K 31/428; A61P 25/14

(52) U.S. Cl. CPC A61K 31/519; A61K 31/381; A61K 31/4045; A61K 31/428; A61P 25/14

(57) ABSTRACT

Compositions and methods are provided for determining protein biomarkers associated with periodic limb movements (PLMs) and/or restless legs syndrome (RLS) in an individual. These protein biomarkers are useful in patient identification and stratification, and also provide drug targets for therapeutic intervention. In some embodiments an individual is treated for PLM and/or RLS by administering a drug that acts on a protein target identified herein.

### CROSS REFERENCE TO OTHER APPLICATIONS

This application claims the benefit of U.S. Provisional Application No. 63/469,620 filed May 30, 2023, the contents of which are hereby incorporated by reference in its entirety.

### GOVERNMENT SUPPORT

This invention was made with Government support under contract HL071515 awarded by the National Institutes of Health. The Government has certain rights in the invention.

### BACKGROUND

Periodic limb movements (PLMs) are episodes of involuntary, repetitive muscle movements affecting the limbs, usually legs, with an estimated prevalence of 4-11%. These generally present as rapid flexions of the big toe, ankle, knee, and/or hip, although they can also affect arms. PLMs increase with age, male gender, obesity, physical inactivity, current smoking, diabetes, antidepressant use, and the presence of restless legs syndrome (RLS).

PLMs are strongly associated with RLS. RLS is characterized by an uncontrollable urge to move extremities in response to uncomfortable/unpleasant sensations. RLS symptoms worsen in the evening or at night and during periods of rest or inactivity. RLS sensations are completely or partially relieved by movement. Presence of PLMs is often used as a supporting feature for an RLS diagnosis as PLMs are considered a motor component of RLS. However, the presence of PLMs and RLS are not mutually interdependent; not all people with RLS have PLMs and vice versa. Thus, although PLMs and RLS are highly correlated, they are two separate phenomena.

Four leading theories have been proposed to explain PLMs: 1) sympathetic nervous system hyperexcitability; 2) disinhibition of spinal cord reflexes by the pyramidal tract or a subcortical region of the brain; 3) dopamine and/or iron dysregulation; and 4) stimulus to the central nervous system generator that may originate from the peripheral nervous system (e.g. peripheral neuropathy). The leading theory underlying the pathophysiology of RLS is central iron deficiency and dopaminergic dysfunction and RLS can be secondary to kidney disease, iron deficiency anemia, and often start during pregnancy, all conditions involving low iron. Identification of biomarkers associated with PLMs and RLS are disclosed herein.

### SUMMARY

Compositions and methods are provided for determining protein biomarkers associated with periodic limb movements (PLMs) and/or restless legs syndrome (RLS) in an individual. These protein biomarkers are useful in patient identification and stratification, and also provide drug targets for therapeutic intervention. In some embodiments an individual is treated for PLM and/or RLS by administering a drug that acts on a protein target identified herein.

A large scale measurement of proteins from multiple sleep clinic patients was performed. All these patients had PLMs measured to generate a PLM index (PLMI), and the presence of RLS determined. Using this panel of proteins, 36 proteins were identified as associated with PLMIs independent of kidney disease. Three proteins are significantly and strongly negatively associated with PLMI: Ferritin light chain, Ferritin Heavy chain and LEAP1 (HAMP; Hepcidin). Other findings of interest include the positive association of PLMI with Cathepsin A levels. Cathepsin A is a pleiotropic lysosomal exopeptidase involved in multiple physiological processes, notably protection of lysosomal beta-galactosidase and neuraminidase activities during lysosomal maturation. In some embodiments, a combination of hepcidin, ferritin, and Cathepsin A provide biomarkers in conjunction with RLS for a diagnosis of PLMs.

Megalin is shown herein to be upregulated in RLS. Megalin is associated with regulation of neurogenesis, calcium binding, vitamin D metabolism, kidney development, amyloid beta clearance, and cardiac development. Megalin is provided as a biomarker and therapeutic target for RLS.

Two proteins shown herein to be elevated in PLM and RLS are Sulfotransferase 1A3 (SULT1A3), and Hepatocyte growth factor-regulated tyrosine kinase substrate (HRS). These proteins are positively correlated with the genetic score of RLS in these subjects, and can be causative of RLS. In some embodiments, Sulfotransferase 1A3 is disclosed as a target for treatment of RLS/PLMI. Inhibitors of Sulfotransferase 1A3 include, for example, tetrahydropterin and other small molecule drugs.

In some embodiments a method is provided for determining whether a patient suffers from PLM and/or RLS. A method can comprise quantitating the level of one, two or three biomarkers selected from hepcidin, ferritin, and Cathepsin A in a patient sample, e.g. a blood sample or derivative thereof; wherein altered levels of these proteins relative to a normal control are indicative of the presence of PLM. Decreased levels of Ferritin and Hepcidin, and increased levels of Cathepsin A are diagnostic for PLM. Other biomarkers for this purpose include, without limitation, SULT1A3, megalin, and HRS. Increased levels of SULT1A3 and HRS are indicative of PLM or RLS. Increased levels of megalin are indicative of RLS.

The biomarker proteins may be quantitated by proteomic profiling by liquid chromatography coupled with tandem mass spectrometry; by immunoassay, by oligonucleotide aptamer-based platforms, such as the SOMAscan assays, etc. In some embodiments the proteomics is combined with a determination of RLS for diagnosis. The data may be analyzed by a computer processor. The processor may be communicatively coupled to a storage memory for analyzing the data. Also described herein is a computer-readable storage medium storing computer-executable program code, the program code comprising code code for storing and analyzing data obtained by the methods of the disclosure.

In some embodiments a method of treatment of RLS and/or PLMI is provided, the method comprising administering an effective dose of an inhibitor of SULT1A3. In some embodiments the inhibitor is tetrahydropterin. In some embodiments the inhibitor is a small molecule. In some embodiments the inhibitor is determined by screening candidate agents for inhibition of SULT1A3 in an in vitro assay.

In other embodiments of the invention a device or kit is provided for the analysis of patient samples. Such devices or kits will include reagents that specifically identify one or more biomarkers indicative of the status of the patient. The reagents can be provided in isolated form, or pre-mixed as a cocktail suitable for the methods of the invention. A kit can include instructions for using the plurality of reagents to determine data from the sample; and instuctions for statistically analyzing the data. The kits may be provided in combination with a system for analysis, e.g. a system implemented on a computer. Such a system may include a software component configured for analysis of data obtained by the methods of the invention.

In an embodiment, the method further comprises selecting a treatment regimen for the patient based on the analysis. In an embodiment, the method further comprises determining a treatment course for the subject based on the analysis. Treatment regimens of interest can include decision-making for proceeding with medication, including without limitation a SULT1A3 inhibitor.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features can be arbitrarily expanded or reduced.

FIG. 1. Volcano plots for proteins associated with the periodic limb movement index (PLMI) for (A) the STAGES cohort and (B) the combined cohort. Red colored circles are proteins negatively associated with PLMI while green colored circles are proteins positively associated with PLMI. Note: Only the top 5 proteins for each direction are labeled with protein names.

FIG. 2. Volcano plots for proteins differentially expressed in restless legs syndrome (RLS) for (A) the STAGES cohort and (B) the combined cohort. Green colored circles are proteins upregulated in RLS.

### DETAILED DESCRIPTION OF THE EMBODIMENTS

All structural and functional equivalents to the features and method acts of the various embodiments described throughout the disclosure that are known or later come to be known to those of ordinary skill in the art are intended to be encompassed by the features described and claimed herein. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “theranosis” refers to the use of results obtained from a diagnostic method to direct the selection of, maintenance of, or changes to a therapeutic regimen, including but not limited to the choice of one or more therapeutic agents, changes in dose level, changes in dose schedule, changes in mode of administration, and changes in formulation. Diagnostic methods used to inform a theranosis can include any that provides information on the state of a disease, condition, or symptom.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers can include expression levels of an intracellular protein or extracellular protein. Markers can also include combinations of any one or more of the foregoing measurements, including temporal trends and differences. Broadly used, a marker can also refer to an immune cell subset.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a blood sample. A sample can include, without limitation, an aliquot of body fluid, whole blood, serum, plasma, synovial fluid, lymphatic fluid, ascites fluid, and interstitial or extracellular fluid. “Blood sample” can refer to whole blood or a fraction thereof. Samples can be obtained from a subject by means including but not limited to venipuncture, biopsy, needle aspirate, lavage, scraping, surgical incision, or intervention or other means known in the art.

Samples may be obtained at one or more time points. Where a sample at a single time point is used, comparison is made to a reference “base line” level for the presence of the activated form of the signaling protein of interest, which may be obtained from a normal control, a pre-determined level obtained from one or a population of individuals, from a negative control for ex vivo activation, and the like.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

Periodic Limb Movement Disorder (PLMs) and Restless Legs Syndrome (RLS) are characterized by abnormal motions of and, for RLS, usually sensations in the lower or upper extremities, which may interfere with sleep. PLMs are more common during middle and older age; >80% of patients with RLS also have PLMS. The mechanism is unclear but may involve abnormalities in dopamine neurotransmission in the central nervous system (CNS). PLMS and RLS can occur in isolation, during drug withdrawal, with use of stimulants, certain antidepressants, or dopamine antagonists, during pregnancy, in patients with chronic renal or hepatic failure, iron deficiency, anemia, diabetes mellitus, a neurologic disorder (eg, multiple sclerosis, Parkinson disease), or other disorders.

In primary RLS, heredity may be involved; more than one third of patients with primary RLS have a family history of it. Additional risk factors may include a sedentary lifestyle, smoking, and obesity. Periodic leg movement disorder is common among people with narcolepsy and rapid eye movement (REM) sleep behavior disorder.

Periodic limb movement disorder is characterized by repetitive (usually every 20 to 40 seconds) twitching or kicking of the lower or upper extremities during sleep. Patients usually complain of interrupted nocturnal sleep or excessive daytime sleepiness. They are typically unaware of the movements and brief arousals that follow and have no abnormal sensations in the extremities. Bed partners may complain about being kicked.

Restless legs syndrome is a sensorimotor disorder characterized by an irresistible urge to move the legs, arms, or, less commonly, other body parts, usually accompanied by paresthesias (eg, creeping or crawling sensations) and sometimes pain in the upper or lower extremities; symptoms are more prominent when patients are inactive or recline and peak in severity around bedtime. To relieve symptoms, patients move the affected extremity by stretching, kicking, or walking. As a result, they have difficulty falling asleep, repeated nocturnal awakenings, or both. Symptoms may be worsened by stress. Episodes may occur occasionally, causing few problems, or daily.

Diagnosis of RLS or PLMs may be suggested by the patient's or bed partner's history. For example, patients with PLMs typically have insomnia, EDS, and/or excessive twitching just before sleep onset or during sleep. Polysomnography may be used to confirm the diagnosis of PLMs, which is usually apparent as repetitive bursts of electromyographic activity. Polysomnography may be also done after RLS is diagnosed to determine whether patients also have PLMs, but polysomnography is not necessary for diagnosis of RLS itself.

Conventional treatment for RLS or PLMs may comprise Pramipexole, ropinirole, a rotigotine patch, or gabapentin enacarbil, plus iron supplements if ferritin is <50 ng/ml. Dopaminergic drugs, although often effective, may have adverse effects such as augmentation (RLS symptoms that worsen before the next drug dose is given and that affect other body parts such as the arms), rebound (symptoms that worsen after the drug is stopped or after the effects of the drug dissipate), nausea, orthostatic hypotension, compulsive activity, and insomnia. Three dopamine agonists, pramipexole, ropinirole, and rotigotine (used as a patch), are effective and have few serious adverse effects other than augmentation. Levodopa/carbidopa may be used, but other drugs, which are less likely to cause augmentation and rebound symptoms, are usually preferred.

Gabapentin may help relieve RLS symptoms and is used when RLS is accompanied by pain. Dosing begins with 300 mg at bedtime and can be increased by 300 mg weekly (maximum dose 900 mg orally 3 times a day). However, gabapentin is not approved for the treatment of RLS. Gabapentin enacarbil, a prodrug of gabapentin, may help relieve RLS symptoms; it is approved for the treatment of RLS. The recommended dose is 600 mg once a day taken with food at about 5 μm. Its most common adverse effects include somnolence and dizziness. It is less likely to cause augmentation than the dopaminergic drugs.

Pregabalin, a nondopaminergic alpha-2-delta ligand, may help relieve RLS symptoms; augmentation is less likely to occur than with pramipexole. Pregabalin may also be useful for RLS accompanied by pain. For RLS, a dose of 300 mg once a day has been used. Dizziness and somnolence are the most common adverse effects. However, use of this drug to treat RLS has not been extensively studied.

Sulfotransferase 1A3/1A4 is a phenol sulfotransferase with thermolabile enzyme activity. SULT1A3 catalyzes the sulfation of phenolic monoamines, such as dopamine, norepinephrine and serotonin, and phenolic and catecholic drugs. SULT1A3 is also called catecholamine sulfotransferase. SULT1A3 prefers endogenous catecholamines as substrates and is highly expressed in the gastrointestinal tract. The refseq for the human protein may be accessed at Genbank, NP_808220; NP_001017390.

Inhibitors of SULT1A3 include, without limitation, tetrahydropterin, NSAIDs and other small molecule drugs. For example, see Cook et al. (2021) JBC VOLUME 296, 100094. Substrates useful in screening of SULT1A3 inhibitors include, for example, those disclosed by Mlynarska-Cieslak, et al. (2022) ACS Chemical Biology 17 (3), 661-669.

The human LEAP1 protein, also known as Hepcidin, is an antimicrobial peptide primarily involved in the regulation of iron homeostasis. It plays a crucial role in the body's defense mechanisms against bacterial infections by limiting iron availability to pathogens. Hepcidin is synthesized in the liver and is responsible for binding to and inducing the degradation of ferroportin, a protein that facilitates the export of iron from cells, thus reducing iron levels in the blood. The gene encoding the LEAP1 protein is called HAMP (Hepcidin Antimicrobial Peptide) and is located on chromosome 19q13.12. The GenBank RefSeq for the human LEAP1 protein is NP_066998.1.

Human Cathepsin A is a lysosomal carboxypeptidase enzyme that plays a critical role in the degradation and processing of various proteins within the lysosome. It is involved in the activation of other lysosomal enzymes, such as beta-galactosidase and neuraminidase, and participates in the breakdown of glycoproteins and glycolipids. Cathepsin A is also associated with the maintenance of the lysosomal function and cellular homeostasis. The gene encoding Cathepsin A is called CTSA and is located on chromosome 20q13.12. The GenBank RefSeq for the human Cathepsin A protein is NP_000299.3.

Human Megalin, also known as low-density lipoprotein receptor-related protein 2 (LRP2), is a large endocytic receptor involved in the absorption and transport of various molecules, including vitamins, hormones, enzymes, and drugs. Megalin plays a crucial role in the reabsorption of these substances in the kidney proximal tubules, preventing their loss in urine, and is also expressed in other tissues such as the brain, lungs, and thyroid gland. The gene encoding Megalin is LRP2, located on chromosome 2q31.1. The GenBank RefSeq for the human Megalin protein is NP_004516.2.

Human Hepatocyte Growth Factor-Regulated Tyrosine Kinase Substrate, commonly known as HRS or HGS, is a protein that plays a pivotal role in intracellular trafficking, signal transduction, and endosomal sorting of ubiquitinated membrane proteins. HRS is involved in the regulation of growth factor receptor degradation, which is crucial for controlling cellular growth and proliferation. It functions as part of the endosomal sorting complex required for transport (ESCRT) machinery, which is essential for the proper sorting of proteins into multivesicular bodies. The gene encoding this protein is HGS, located on chromosome 17q25.3. The GenBank RefSeq for the human Hepatocyte Growth Factor-Regulated Tyrosine Kinase Substrate protein is NP_004707.1.

### Pharmaceutical Compositions

Therapeutic compounds, e.g. inhibitors of SULT1A3, can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4 °C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the agent is formulated with a second agent in a pharmaceutically acceptable excipient(s).

The subject formulations can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

Each of the active agents can be provided in a unit dose of from about 0.01 μg, 0.05 μg, 0.1 μg, 0.5 μg, 1.0 μg, 5.0 μg, 10 μg, 50 μg, 100 μg, 5000 μg, 1 mg, 5 mg, 10 mg, 50 mg, 100 mg, 250 mg, 500 mg, 750 mg or more.

The agent, e.g. a SULT1A3 inhibitor, may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Methods

In some embodiments a method is provided for determining whether a patient suffers from PLM and/or RLS. A method can comprise quantitating the level of one, two or three biomarkers selected from hepcidin, ferritin, and Cathepsin A in a patient sample, e.g. a blood sample or derivative thereof; wherein altered levels of these proteins relative to a normal control are indicative of the presence of PLM. Decreased levels of ferritin and hepcidin, and increased levels of cathepsin A are diagnostic for PLM. Other biomarkers for this purpose include, without limitation, SULT1A3, megalin, and HRS. Increased levels of SULT1A3 and HRS are indicative of PLM or RLS. Increased levels of megalin are indicative of RLS.

In some embodiments, the methods comprise obtaining a biological sample for analysis. Samples can be obtained from the tissues or fluids of an individual. For example, samples can be obtained from whole blood, tissue biopsy, serum, etc. Other sources of samples are body fluids such as lymph, cerebrospinal fluid, and the like. Also included in the term are derivatives and fractions of such cells and fluids. The biological sample may be blood, or a blood-derived sample. In some embodiments the sample is plasma. Samples can be obtained once or multiple times from an individual. Multiple samples can be obtained at different times from the individual. In some embodiments, the blood sample is a peripheral blood sample.

When samples are obtained as a series, the samples can be obtained at fixed intervals, at intervals determined by the status of the most recent sample or samples or by other characteristics of the individual, or some combination thereof. It will be appreciated that an interval may not be exact, according to an individual's availability for sampling and the availability of sampling facilities, thus approximate intervals corresponding to an intended interval scheme are encompassed by the invention.

Samples may be obtained at one or more time points. Where a sample at a single time point is used, comparison is made to a reference “base line” level for the presence of the biomarkers of interest, which may be obtained from a normal control, a pre-determined level obtained from one or a population of individuals, from a negative control for ex vivo activation, and the like.

Multiparametric analysis of biological samples obtained from an individual is used to obtain a determination of protein biomarkers indicative of RLS and/or PLM, which changes include, without limitation, altered levels of one or more of hepcidin, ferritin, cathepsin A, SULT1A3, megalin, and HRS. Increased levels of SULT1A3 and HRS are indicative of PLM or RLS. Increased levels of megalin are indicative of RLS. Decreased levels of hepcidin and ferritin are indicative of PLM. Increased levels of cathepsin A are indicative of PLM.

The biomarker proteins may be quantitated by proteomic profiling by liquid chromatography coupled with tandem mass spectrometry; by immunoassay, by oligonucleotide aptamer-based platforms, such as the SOMAscan assays, etc. In some embodiments the proteomics is combined with a determination of RLS for diagnosis. The data may be analyzed by a computer processor. The processor may be communicatively coupled to a storage memory for analyzing the data. Also described herein is a computer-readable storage medium storing computer-executable program code, the program code comprising code code for storing and analyzing data obtained by the methods of the disclosure.

The differential presence of these markers is shown to provide for diagnostic evaluations to detect individuals having RLS and/or PLM; and to stratify for clinical purposes the presence of RLS in the absence of PLM, e.g. by determination of increased levels of megalin. Detection can utilize one or a panel of specific binding members, e.g. a detection specific for one, two, three, four, five, or more markers.

Levels of a biomarker, e.g. in plasma, may be altered, i.e. reduced or increased in level, by more than 1.25-fold, more than 1.5-fold, more than 1.75-fold, more than 2-fold, more than 2.25-fold, more than 2.5-fold, more than 2.75-fold, more than 3-fold, or more relative to a normal control sample. Levels of a biomarker, e.g. in plasma, may be altered, i.e. reduced or increased in level, by at least 1.25-fold, at least 1.5-fold, at least 1.75-fold, at least 2-fold, at least 2.25-fold, at least 2.5-fold, at least 2.75-fold, at least 3-fold, or more, relative to a normal control sample.

Individuals diagnosed with RLS and/or PLM; or stratified into RLS and/or PLM, can be treated accordingly, for example with an inhibitor of SULT1A3.

In some embodiment, the methods of the invention include the use of liquid handling components for analysis of biomarkers. The liquid handling systems can include robotic systems comprising any number of components. In addition, any or all of the steps outlined herein can be automated; thus, for example, the systems can be completely or partially automated. As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this can be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

A signature pattern of biomarker expression can be generated from a biological sample using any convenient protocol, for example as described below. The readout can be normalized, a mean, average, median or the variance or other statistically or mathematically-derived value associated with the measurement. The marker readout information can be further refined by direct comparison with the corresponding reference or control pattern. A pattern can be evaluated on a number of points: to determine if there is a statistically significant change at any point in the data matrix relative to a reference value; whether the change is specific for one or more physiological states, and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a prognosis regarding the phenotype of the patient from which the sample was obtained/derived. Additionally, a reference or control signature pattern can be a signature pattern that is obtained from a sample of a patient known to have a normal pregnancy.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern can be compared to a positive and negative reference profile to obtain confirmed information regarding whether the patient has the phenotype of interest.

In order to identify profiles that are indicative of RLS and/or PLM, a statistical test can provide a confidence level for a change in the level of markers between the test and reference profiles to be considered significant. The raw data can be initially analyzed by measuring the values for each marker, usually in duplicate, triplicate, quadruplicate or in 5-10 replicate features per marker. A test dataset is considered to be different than a reference dataset if one or more of the parameter values of the profile exceeds the limits that correspond to a predefined level of significance.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). This analysis algorithm is currently available as a software “plug-in” for Microsoft Excel know as Significance Analysis of Microarrays (SAM). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is. The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation. Alternatively, any convenient method of statistical validation can be used.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling's T2 statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logistic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

These tools and methods can be applied to several classification problems. For example, methods can be developed from the following comparisons: i) all cases versus all controls, ii) all cases versus nonresponsive controls, iii) all cases versus responsive controls.

In a second analytical approach, variables chosen in the cross-sectional analysis are separately employed as predictors. Given the specific outcome, the random lengths of time each patient will be observed, and selection of proteomic and other features, a parametric approach to analyzing responsiveness can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

In addition the Cox models can be used, especially since reductions of numbers of covariates to manageable size with the lasso will significantly simplify the analysis, allowing the possibility of an entirely nonparametric approach to survival.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

Objectives: We used a high-throughput assay of 5,000 plasma proteins to identify biomarkers associated with periodic limb movements (PLMs) and restless legs syndrome (RLS) in adults.

Methods: Participants (n=1410) of the Stanford Technology Analytics and Genomics in Sleep (STAGES) study had blood collected, completed a sleep questionnaire and underwent overnight polysomnography with scoring of PLMs. An aptamer-based array (SomaScan) was used to quantify 5,000 proteins in plasma. A second cohort (n=697) that had serum assayed using a previous iteration of SomaScan (1,300 proteins) was used for replication and in a combined analysis (n=2107). A 5% False Discovery Rate (FDR) was used to assess significance.

Results: Multivariate analyses in STAGES identified 68 proteins associated with the PLM Index (PLMI) after correction for multiple testing (i.e., base model). Most significantly decreased proteins were iron-related and included Hepcidin (LEAP-1), Ferritin, and Ferritin light chain. Most significantly increased proteins included RANTES, Cathepsin A and SULT 1A3. Of 68 proteins significant in the base model, 17 were present in the 1,300 panel, and 15 of 17 replicated. The most significant proteins in the combined model were Hepcidin (LEAP-1), Cathepsin A, Ferritin, and RANTES. Exploration of proteins in RLS vs non RLS identified Cathepsin Z, Heme oxygenase 2 (HO-2), Interleukin-17A (upregulated in the combined cohort) and Megalin (upregulated in STAGES only), although results were less significant than for proteins associated with PLMI.

Conclusions: These results confirm the association of PLM with low iron status and suggest involvement of catabolic enzymes in PLM/RLS.

In this study, we hypothesized that high-throughput proteomics could help differentiate RLS and PLMs. Further, we explored if proteomic biomarkers of PLMs could have diagnostic value, as have been evaluated in other sleep disorders (e.g., sleep apnea). To date, only four studies have evaluated proteomic markers of RLS and found associations with proteins linked to inflammation, immune response, and iron deficiency. Until now, however, we were unable to locate a study evaluating large scale proteomic biomarkers of PLMs. In the present study, we used an array of ~5,000 plasma proteins to identify biomarkers associated with PLMs and RLS in 1,410 adults. A second cohort using ~1,300 serum proteins in 697 patients with sleep disorders was used for replication.

### Methods

Stanford Technology Analytics and Genomics in Sleep (STAGES) Study Cohort. The Stanford Technology Analytics and Genomics in Sleep (STAGES) study included patients recruited between 2018 and 2020 across 11 different sleep clinics. Briefly, participants were evaluated by a sleep specialist, completed an overnight PSG, completed a sleep symptom questionnaire (the Alliance Sleep Questionnaire, ASQ) and provided a blood sample. Ethical Review Boards at each institution approved all study procedures and all participants provided informed consent.

Polysomnography in STAGES. Participants completed a Level 1 nocturnal PSG in a sleep lab. PLMs were recorded using surface EMG on one or both lower extremities. PLMs were scored using a previously validated, automated PLM detection algorithm, which has been applied in other studies of PLM clinical outcomes. The algorithm matches the 2016 World Association of Sleep Medicine criteria for scoring a PLM, with minor improvements. The primary outcome of interest was the PLM index (PLMI) (i.e., number of PLMs per hour of sleep). Patients with a PSG duration of two hours or more were included in final analyses.

Alliance Sleep Questionnaire in STAGES. The ASQ is a comprehensive sleep disorders questionnaire designed to collect standardized subjective sleep and sleep disorder data. It uses branching logic in a modular fashion to guide participants through a comprehensive set of questions covering topics such as current sleep symptoms, sleep habits and schedule, and medical history. The ASQ takes ~20 minutes to complete for a healthy user and 45 minutes for a patient with complex sleep issues. The ASQ was completed by participants the day of or within 4 weeks of the PSG.

Restless legs syndrome in STAGES. The ASQ includes items to screen for the presence of RLS based on the International Restless Legs Syndrome Study Group Diagnostic Criteria. The ASQ has been shown to have moderate sensitivity and strong specificity for identifying patients with RLS compared to a physician diagnosis. Detailed information about RLS classification based on item responses can be found in Supplementary Information, including the exclusion of common mimics. Briefly, a person was considered to have RLS symptoms if they provided affirmative responses to diagnostic criteria, had symptoms on two or more days/week with moderate-to-severe RLS-related distress, and were untreated for RLS. A person was considered not to have RLS (i.e., control) if they did not meet diagnostic criteria, had no previous RLS diagnosis, and reported no RLS symptoms or RLS-related distress. We excluded people with RLS who were on treatment from final analyses.

Blood Sampling in STAGES. Blood samples were collected around the time of the scheduled PSG, either the evening before or the morning after the PSG, or within four weeks of the PSG. As time of sampling was not standardized and protein expression can be sensitive to circadian and/or diurnal systems, the time of blood draw was dichotomized into samples collected in the morning (i.e., 0:00-11:59) or evening (i.e., 12:00-23:59) and included as a covariate. Samples were shipped from study sites overnight at room temperature, in most cases taking 1-2 days to arrive to Stanford. These were then processed for EDTA plasma and DNA extraction. As there was variability in time samples remained at room temperature (i.e., time to blood processing), this was included in all analyses as a salient covariate, as in our prior study. Time to processing was calculated as the total number of hours between time of blood sample collection to time plasma was frozen, which was then log-normalized. Extreme outliers were removed (n=7) to reduce variability.

Stanford Sleep Cohort (SSC). We sought to extend and replicate findings obtained with STAGES using the Stanford Sleep Cohort (SSC) described previously. In this study, 1,070 participants ages 18-91 who completed an overnight PSG at the Stanford Sleep Clinic from 1999-2005 were enrolled, provided a blood sample, and completed a short questionnaire. Blood samples were collected the morning following overnight PSG and allowed to clot for 30-60 minutes. Serum was then aliquoted and stored at −80 °C until it was shipped to SomaLogic for protein quantification. Of note, serum samples from this study, athough immediately processed after collection, were frozen for a mean of 11.6 years before being assayed (i.e., time frozen between blood draw to SomaScan assay). Time frozen at −80 °C was included as a covariate in analyses. As with STAGES, PSG studies were scored using a previously validated, automated PLM detection algorithm. A total of 697 participants had relevant data and were included in analyses.

Restless Legs Syndrome in SSC. The questionnaire included two items assessing the presence of RLS including “RLS at Sleep Onset” and “RLS Disturbed Sleep” for which participants responded on a Likert-type scale ranging from 1 (Never) to 5 (Always). Participants were considered to have RLS if they responded “Usually” or “Always” to both items. Participants were considered without RLS symptoms if they responded “Never” or “Rarely” to both items.

Protein Quantification. Plasma from STAGES and serum from SSC were assayed using the SomaScan aptamer-based multiplexed platform (SomaLogic Inc., Boulder, CO), which utilizes aptamers and hybridization to quantify proteins from small amounts of human tissues. This platform has been used to identify clinical indicators of health conditions or risk. The platform was designed so that protein levels can be measured over a range of concentrations (extended dynamic range). It includes both extracellular and intracellular proteins (with soluble domains of membrane proteins). SomaLogic also conducted data quality controls at the sample and protein level to adjust for variability. The present study utilized the most normalized output provided by SomaLogic based on our sample distributions. Plasma samples from STAGES used the SomaScan 5K platform of 4,985 proteins after removal of non-human proteins (e.g., mouse). Serum samples from SSC used the SomaScan 1.3K platform of 1,317 proteins.

GWAS Principal Components. As genetic ancestry is associated with changes in protein levels, the first five principal components (PCA) from a GWAS analysis of the same samples genotyped using Affymetrix PMRA array were included as covariates. Quality control was conducted to ensure no duplicated patients were present based on inheritance by descent using KING. Variants and samples with missing call rates lower than 0.1, and variants with minimum allele frequency lower than 0.05 were excluded. Imputations calls with R²≥0.9 were selected for PCA using PLINK.

Statistical Analyses. Data were analyzed using Jupyter Notebook with Python and the statsmodels module. We examined differences in demographic and clinical characteristics between participant sub-groups (e.g., RLS and controls) with Mann Whitney-U tests and Chi-squared tests for dichotomous variables, whereby an a priori alpha level of 0.05 indicated a significant difference between groups.

Multivariate Proteomic Analyses in Plasma Samples. We conducted linear regression models in the STAGES cohort with log-normalized protein expression as the dependent variable and log-normalized PLMI+1 as the independent variable. Covariates included age, sex, BMI, log-normalized time to blood processing, blood draw period (i.e., morning or evening), study site, and the first five GWAS principal components (i.e., “Base Model”). We also conducted models with the presence of RLS as an independent variable. As PLMs and RLS are associated with kidney dysfunction, we included log-normalized expression of Cystatin C, an indicator of kidney function, as another covariate in additional models. The Benjamini and Hochberg method for controlling the false discovery rate (FDR) was applied with an a priori p-value of 0.05 for statistically significant predictors of protein expression in all models. As STAGES samples were collected in 11 different sites, time to blood processing varied in our sample. To account for this, we examined the effect of blood processing time in the STAGES samples in comparison to 224 samples processed immediately after collection. To facilitate interpretation of our findings, p-values, and beta coefficients for the effect of time to processing are presented in supplementary materials alongside multivariate results.

Pathway Analysis. The ToppFun module of the ToppGene suite (Cincinnati, OH, USA) was utilized for biological pathway enrichment analysis for all 5% FDR significant protein sets. The ToppGene Suite is a free web portal for executing candidate gene/protein prioritization using functional annotations or network analysis. The NCBI Entrez gene IDs of all 5% FDR significant proteins were input into the ToppFun module and results are presented based on significant pathways/functions identified by ToppFun.

Replication in Serum Samples of the SSC. As the SSC utilized the SomaScan 1.3K platform, these analyses were conducted in 1,282 proteins present in both the 1.3K and 5K panels. Log-normalized protein expression was the dependent variable and covariates included age, sex, BMI, log-normalized time to blood processing, age of samples, blood draw period, GWAS principal components, and study (i.e., STAGES or SSC). Correction for multiple testing (FDR) was applied with an a priori p-value of 0.05 for statistically significant predictors of protein expression in all models.

Validation of Protein Markers with Machine Learning. To predict PLMI, the Python Scikit-learn LassoCV library was used to train least absolute shrinkage and selection operator (i.e., Lasso) linear models with iterative fitting along a regularization path and 5-fold cross-validation for optimal model selection. Hyperparameters of the models were also fine-tuned via a 5-fold cross-validation. Analyses were conducted in STAGES-only samples and in the combined STAGES-SSC samples using a 70% training-30% testing split each time. In STAGES, 931 participants were used for training (mean PLMI=11.2) and 399 (mean PLMI=11.1) for testing. In the combined cohort, 1,253 (mean PLMI=11.5) participants were used for training and 537 (mean PLMI=11.9) for testing. In both cases, five models were compared: (1) study site; (2) study site and demographics (i.e., age, sex, BMI); (3) study site, demographics, and top proteins identified in multivariate analyses; (4) study site, demographics, and RLS classification (i.e., RLS or control); and (5) study site, demographics, top proteins, and RLS classification. R², mean absolute error (MAE), and root-mean-square error (RMSE) values are presented to estimate Lasso model performances. Preprocessing included log-normalization of relative protein expression, followed by conversion of continuous variables to a standardized scale using the zero mean with unit variance method. All models used the same preprocessing, test-train split, and LassoCV approach for comparison.

### Results

Sample Characteristics. Demographic and clinical information of participants. STAGES (n=1,410) patients had an average PLMI of 11.0±18.1/hour with most participants (n=1119; 79%) having at least one PLM count during the night (range: 4 to 998) and 21% having a PLMI>15 (54% males), shown in Table 1. In the SSC, average PLMI was 15.4±25.3/hour and 28% having a PLMI≥15 (66% male). In STAGES, 7% (n=102) screened positive for RLS symptoms. Subjects with RLS had higher BMI (p=0.009), total PLM counts (p=6.25×10⁻⁵), PLMI (p=7.44×10⁻⁵), and 33% had PLMI ≥15 vs. 16% in controls (p=4.53×10⁻⁵). Further, 27% (n=28) of those with RLS reported a family history of RLS. In the SSC, 7% of (n=47) screened positive for RLS. These were more female (60%, p=0.015), had higher total PLM count (p=0.036), higher PLMI (p=0.017), and a higher proportion of subjects with PLMI ≥15 (p=0.015) versus controls. These characteristics are consistent with presentation of RLS and support an accurate classification of RLS in these cohorts.

TABLE 1
Demographic and clinical characteristics
of participants included in

[symbol]

| | [symbol] | [symbol] | [symbol] | p |
|---|---|---|---|---|
| Age (years) | [symbol] | [symbol] | [symbol] | [symbol] |
| Sex (female) | [symbol] | [symbol] | [symbol] | [symbol] |
| BMI (kg/m [symbol] ) | [symbol] | [symbol] | [symbol] | [symbol] |
| Race | | | | [symbol] |
| Caucasian/White | [symbol] | [symbol] | [symbol] | |
| Asian | [symbol] | [symbol] | [symbol] | |
| Two or More | [symbol] | [symbol] | [symbol] | |
| Other (Latino/Black/African American/American | [symbol] | [symbol] | [symbol] | |
| Indian/Alaska Native/Pacific Islander) | | | | |
| Ethnicity (Hispanic Origin; yes) | | [symbol] | [symbol] | |
| Total sleep time (hours) | [symbol] | [symbol] | [symbol] | [symbol] |
| Sleep efficiency (%) | [symbol] | [symbol] | [symbol] | [symbol] |
| Total PLM [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| Hypertension | [symbol] | [symbol] | [symbol] | [symbol] |
| High cholesterol | [symbol] | [symbol] | [symbol] | [symbol] |
| Diabetes II | | [symbol] | [symbol] | |
| Blood draw period | | | | [symbol] |
| AM (0:00-11:59) | [symbol] | [symbol] | [symbol] | |
| PM (12:00-23:59) | [symbol] | [symbol] | [symbol] | |
| [symbol] | | [symbol] | [symbol] | |
| Age of samples (years) | | [symbol] | [symbol] | |

Note:

[symbol]

[symbol]

[symbol]

[symbol]

indicates data missing or illegible when filed

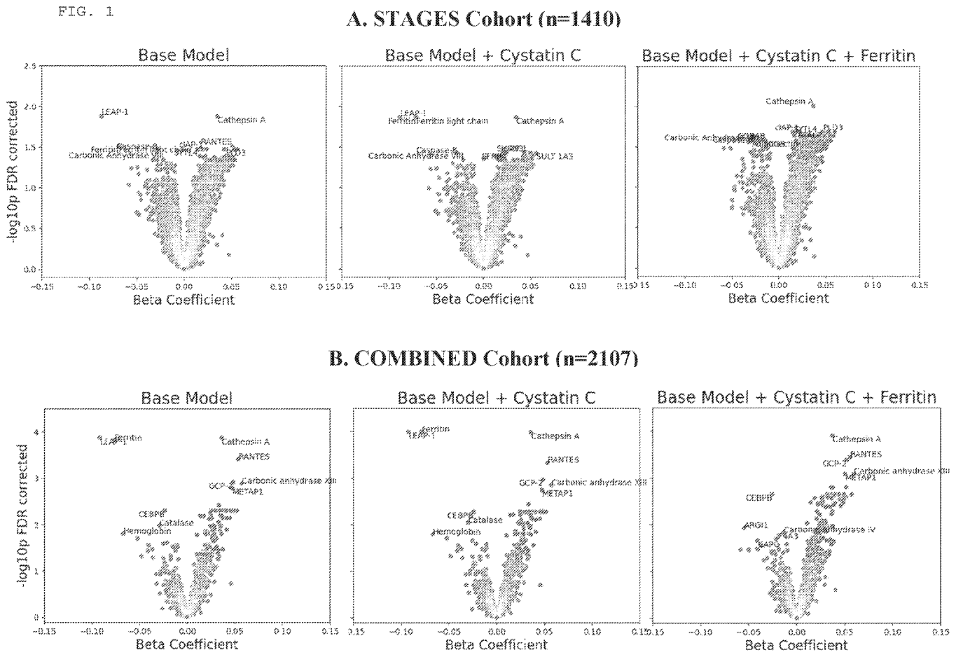

Proteins Associated with the PLMI in STAGES. Proteins associated with PLMI in STAGES (n=1410) are presented in FIG. 1A. Multivariate analyses identified 68 proteins (7 negatively and 61 positively) associated with PLMI after FDR correction (FIG. 1A left). Proteins negatively associated with PLMI include Hepcidin (LEAP-1), Ferritin, Ferritin light chain, Caspase-8, Selenoprotein H (SELH), Adiponectin, and Carbonic Anhydrase VIII. These negatively associated proteins belong to pathways involving iron metabolism, iron uptake and transport, hepcidin production, and apoptosis. Proteins most positively associated with PLMI include lysosomal protective protein Cathepsin A, C-C motif chemokine 5 (RANTES), Sulfotransferase 1A3 (SULT 1A3), Carbonic anhydrase XIII, Phospholipidase D3 (PLD3), SH3 and multiple ankyrin repeat domains protein 3 (SHAN3), and Secreted frizzled-related protein 4 (SFRP4). Pathways upregulated involved chemokine receptors, NOD-like receptor signaling, and innate immunity.

Inclusion of Cystatin C (to control for kidney function) to the model (FIG. 1A middle) reduced the number of associated proteins to 36 (5 negative and 31 positive), although the top 30 proteins of the base model remained the same. Inclusion of Cystatin C and Ferritin to the base model (FIG. 1A right) increased the number of associated proteins to 314 (31 negative and 283 positive), with all proteins of the base model with/without Cystatin C remaining significantly associated, apart from Ferritin, Ferritin light chain, and Hepcidin (i.e., iron-related proteins). Pathway analyses demonstrated similar pathways to the base model. Further, proteins significantly associated with PLMI in the base model remained significant despite inclusion of C-reactive protein (i.e., marker of inflammation) and presence of hypertension or high cholesterol. The inclusion of the presence of RLS or medications known to influence RLS as covariates in the base model had negligible effect on the beta coefficients for proteins associated with PLMI in the STAGES cohort. Of note, inclusion of Cathepsin A as the top protein associated with PLMI in addition to Cystatin C and Ferritin resulted in no remaining proteins significantly associated with PLMI, suggesting that a combination of proteins related to iron deficiency (represented by Ferritin), kidney dysfunction (Cystatin C) and another pathway labelled by Cathepsin A summarizes most of the signal.

Replication of Periodic Limb Movement Index Proteins with Serum Samples. In the SSC alone (n=697), no proteins were associated with PLMI after FDR correction. Of 68 significant proteins in the STAGES base model, only 17 were present in the 1.3K panel and available for replication in the combined dataset. Results from multivariate analyses in the combined sample (n=2107) including both the STAGES cohort and the SSC cohort are presented in FIG. 1B. In the base model, 15 of the 17 overlapping proteins associated with PLMI in STAGES replicated in SSC; these were also associated with PLMI in the combined analysis after FDR correction. Proteins included Hepcidin (LEAP-1), Cathepsin A, Ferritin, RANTES, Carbonic anhydrase XIII, Methionine aminopeptidase 1 (METAP1), C-X-C motif chemokine 6 (GCP-2), Growth-regulated alpha protein (Gro-a), 3-hydroxyacyl-CoA dehydrogenase type-2 (ERAB), Sphingosine kinase 1, and Nascent polypeptide-associated complex subunit alpha (NACA). Pathways related to these proteins were similar to those obtained in STAGES.

Validation of Protein Markers with Machine Learning. Because iron deficiency (e.g., Ferritin), kidney dysfunction (Cystatin C) and another pathway marked by Cathepsin A best summarized the signal, we next used Lasso including these proteins to predict PLMI (Table 2). For both STAGES (n=1,330) and the combined sample (n=1,790), model 5 (i.e., study site/study, demographics, proteins, RLS) had the strongest performance (STAGES R²=0.131, combined R²=0.116) followed by model 4 incorporating RLS without proteins (STAGES R²=0.117, combined R²=0.106) and model 3 incorporating proteins without RLS (STAGES R²=0.100, combined R²=0.087). These results indicate that PLMI prediction is modestly improved beyond presence of RLS and demography by measuring these proteins.

TABLE 2

[symbol]

| | [symbol] | | | | [symbol] | | | |
|---|---|---|---|---|---|---|---|---|
| [symbol] | Model [symbol] | R² | [symbol] | [symbol] | model [symbol] | R² | [symbol] | [symbol] |
| 1 | [symbol] | −0.001 | [symbol] | [symbol] | [symbol] | [symbol] | [symbol] | [symbol] |
| 2 | [symbol] | [symbol] | [symbol] | [symbol] | [symbol] | [symbol] | [symbol] | [symbol] |
| 3 | [symbol] | [symbol] | [symbol] | [symbol] | [symbol] | [symbol] | [symbol] | [symbol] |
| 4 | [symbol] | [symbol] | [symbol] | [symbol] | [symbol] | [symbol] | [symbol] | [symbol] |
| 5 | [symbol] | [symbol] | [symbol] | [symbol] | [symbol] | [symbol] | [symbol] | [symbol] |

[symbol]

[symbol]

indicates data missing or illegible when filed

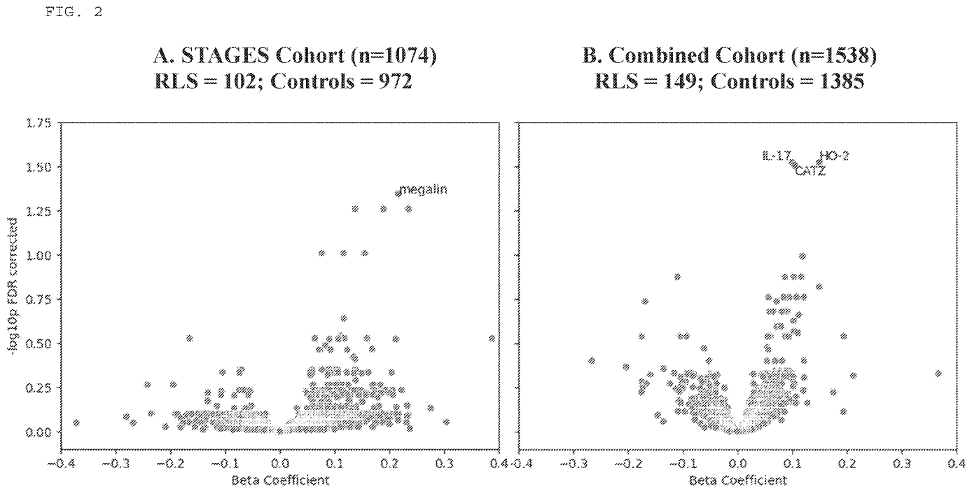

Proteins Associated with Restless Legs Syndrome. The summary of differentially expressed proteins (DEPs) in RLS after FDR correction is graphically illustrated in FIG. 2. In STAGES only, one protein, low-density lipoprotein receptor-related protein 2 (Megalin), was differentially expressed in RLS vs controls after FDR correction. This protein showed a 3.98-fold increase in people with RLS (n=102; 7%) compared to controls (n=972). Megalin, one of the largest plasma membrane proteins, is an endocytic “scavenging” receptor. It is associated with regulation of neurogenesis, calcium binding, vitamin D metabolism, kidney development, amyloid beta clearance, and cardiac structure development. The significance of this protein was unchanged by the inclusion of Cystatin C and Ferritin as covariates in the model.

In SSC alone, no proteins were differentially expressed in RLS (n=47; 7% of 413) compared to controls. Unfortunately, Megalin was not present in the 1.3K panel and, therefore unavailable for replication. In the combined dataset (n=1538) including STAGES and SSC, three proteins were increased in RLS vs controls after FDR (Supplementary Table S16.B): Cathepsin Z (CATZ, 1.13-fold), Heme oxygenase 2 (HO-2, 1.28-fold), and Interleukin-17A (IL-17, 1.42-fold). These proteins are associated with pathways involving heme degradation, interleukin signaling, cytokine networks, iron uptake/transport, innate immune system, and apoptosis. Significance was unchanged after inclusion of Cystatin C and Ferritin as covariates.

### Discussion

This study identified two sets of pathways associated with PLMs. Most strikingly, we found that PLMI is associated with lower levels of ferritin and hepcidin (LEAP-1), proteins that correlate with each other and are involved in iron metabolism. Low ferritin has previously been associated with PLMs in the Wisconsin Sleep Cohort, a population-based study. This extends this finding in two other cohorts totaling 2,000 individuals further confirming the role of iron dysfunction in PLMs. These results are consistent with PLM correlations with iron deficiency biomarkers (ferritin concentration<50 μg/L, UIBC, TIBC, and serum transferrin concentration, but not serum iron or TSAT) in a recent sample of 100 subjects with RLS although the correlations were not found in another sample of similar size.

Ferritin is a carrier of iron in the blood and is used intracellularly to store iron. Low levels of Ferritin typically indicate depleted iron stores, although increased ferritin can also reflect inflammation. Hepcidin, a liver-produced protein, is a key regulator of the entry of iron into the circulation in mammals. In contrast to ferritin, hepcidin is regulated both by anemia and hypoxia, and this may explain why in a prior study increased hepcidin was found with continuous positive airway pressure therapy in patients with obstructive sleep apnea. As for ferritin, hepcidin levels can be increased by inflammation, and low levels are associated with anemia. Although ferritin and hepcidin are correlated in our study, as shown in other populations, these molecules do not reflect the same aspect of iron metabolism and the fact both are low in PLM likely reflects lower circulating and stored iron.

Although there are strong data suggesting RLS can be associated with iron depletion, and may be reversed by intravenous iron, data are controversial for primary RLS. Most notably, genetic studies have not found overlaps with genetic factors associated with biomarkers of iron depletion or ferritin levels, arguing against causation. In our study, lower ferritin and hepcidin, associated with PLMs, were not associated with RLS. These variable findings are consistent with other studies that have studied small samples of clinically diagnosed RLS patients. As mentioned above, one study in 100 subjects found a correlation of low iron status or low hepcidin levels with high PLMI but not RLS severity in patients with RLS. Another study found no association of iron status with RLS and others the opposite. Similarly, although some results have suggested decreased ferritin in the CSF and brain of RLS patients (as opposed to peripheral levels), other studies could not replicate these findings.

A novel finding of this study is the positive association of PLMI with Cathepsin A levels. Cathepsin A is a pleiotropic lysosomal exopeptidase involved in multiple physiological processes, notably protection of lysosomal beta-galactosidase and neuraminidase activities during lysosomal maturation. As a result, Cathepsin-A mutations are associated with galactosialidosis and GM1 gangliosidosis. In addition, Cathepsin A is involved in the degradation and maturation of many neuropeptides, Rich thus why PLMI is associated with levels of Cathepsin A could be the result of multiple effects. Cathepsin A levels are also correlated with those of many proteins, including RANTES/CCL5 an inflammatory cytokine, thus this may also reflect correlation, not causation.

We found upregulation of CATZ, HO-2, IL-17 (combined only) and Megalin (STAGES only) in RLS. Of note, CATZ and HO-2 were upregulated in RLS within the STAGES cohort; however, not significantly after FDR correction. CATZ is a protein associated with immune processes and cancer as well as inflammatory gastric disease and may be related to dopamine neuron death. HO-2 is involved in heme catabolic processes and oxidation as well as iron homeostasis and IL-17 is an effector cytokine of innate and adaptive immune pathways. The upregulation of these pathways overlap with RLS pathology in that central dopamine dysfunction is a leading theory for the pathogenesis of RLS. Further, RLS is frequently associated with inflammatory and immune disorders, including Multiple Sclerosis, Rheumatoid Arthritis, Celiac Disease and irritable bowel syndrome.

The four proteins significantly upregulated in the present study were not identified in prior proteomic studies of RLS. Although not significant after FDR correction, Kininogen-1 (KNG1) was upregulated and Alpha-2-HS-glycoprotein (AHSG) and Complement C3 (C3) were downregulated in RLS in both STAGES and combined analyses, proteins that have been differentially expressed in RLS in previous reports. Additionally, endoplasmic reticulum membrane protein complex subunit 1 (EMC1) was upregulated in the STAGES cohort before FDR correction, consistent with another recent study. One of the most prominent findings in previous proteomic studies was the differential expression of vitamin D-binding protein (GC), although, those studies were inconsistent in the direction of association with RLS. Notwithstanding that our study did not demonstrate such an association with that protein or the vitamin D3 receptor (VDR) protein, we did find megalin to be upregulated in RLS. Megalin is associated with regulation of neurogenesis, calcium binding, vitamin D metabolism, kidney development, amyloid beta clearance, and cardiac development, pathways previously implicated in RLS.

The present study conducted the largest examination of proteins to date in adults with PLMs and RLS. Our results show that PLMs are associated with iron deficiency and with changes in proteolytic enzyme activity such as Cathepsin A. Results in RLS were more variable, a finding likely due to the smaller number of subjects with RLS. These findings confirm the connection between iron dysfunction and PLMs, and perhaps less strongly RLS. Machine learning models suggest that that RLS classification in combination with proteins will give the strongest prediction of PLMI, although RLS classification alone was stronger than proteins alone. Collectively, these findings support use of hepcidin, ferritin, and Cathepsin A as biomarkers in conjunction with RLS in the ascertainment of PLMs. Considering the link between these conditions and increased risk of cardiovascular disease, hypertension, stroke, depression and anxiety, this study further clarifies the pathobiology of PLMs and improves our understanding of their biological and clinical relevance. Further, measurement of these biomarkers may inform additional therapeutic strategies in PLM and RLS management.

### REFERENCES

Hornyak M, Feige B, Riemann D, Voderholzer U. Periodic leg movements in sleep and periodic limb movement disorder: prevalence, clinical significance and treatment. Sleep Med Rev. 2006; 10 (3): 169-177. doi:10.1016/j.smrv.2005.12.003
Szentkirályi A, Stefani A, Hackner H, et al. Prevalence and associated risk factors of periodic limb movement in sleep in two German population-based studies. Sleep. 2019; 42 (3). doi:10.1093/sleep/zsy237
Hamilton-Stubbs P, Walters A S. Periodic Limb Movements. In: Kompoliti K, Metman L V, eds. Encyclopedia of Movement Disorders. Oxford: Academic Press; 2010:444-449.
Didato G, Di Giacomo R, Rosa G J, Dominese A, de Curtis M, Lanteri P. Restless Legs Syndrome across the Lifespan: Symptoms, Pathophysiology, Management and Daily Life Impact of the Different Patterns of Disease Presentation. Int J Environ Res Public Health. 2020; 17 (10). doi:10.3390/ijerph17103658
Moore Ht, Winkelmann J, Lin L, Finn L, Peppard P, Mignot E. Periodic leg movements during sleep are associated with polymorphisms in BTBD9, TOX3/BC034767, MEIS1, MAP2K5/SKOR1, and PTPRD. Sleep. 2014; 37 (9): 1535-1542. doi:10.5665/sleep.4006
Didriksen M, Nawaz M S, Dowsett J, et al. Large genome-wide association study identifies three novel risk variants for restless legs syndrome. Commun Biol. 2020; 3 (1): 703. doi:10.1038/s42003-020-01430-1
Edelson J L, Schneider L D, Amar D, et al. The Genetic Etiology of Periodic Leg Movement in Sleep. Sleep. 2022. doi:10.1093/sleep/zsac121
Ambati A, Ju Y E, Lin L, et al. Proteomic biomarkers of sleep apnea. Sleep. 2020; 43 (11). doi:10.1093/sleep/zsaa086
Cederberg K L J, Hanif U, Peris Sempere V, et al. Proteomic Biomarkers of the Apnea Hypopnea Index and Obstructive Sleep Apnea: Insights into the Pathophysiology of Presence, Severity, and Treatment Response. Int J Mol Sci. 2022; 23 (14). doi:10.3390/ijms23147983
Bellei E, Monari E, Ozben S, et al. Discovery of restless legs syndrome plasmatic biomarkers by proteomic analysis. Brain Behav. 2018; 8 (10):e01062. doi:10.1002/brb3.1062
Patton S M, Cho Y W, Clardy T W, Allen R P, Earley C J, Connor J R. Proteomic analysis of the cerebrospinal fluid of patients with restless legs syndrome/Willis-Ekbom disease. Fluids Barriers CNS. 2013; 10 (1): 20. doi:10.1186/2045-8118-10-20.
Shin J W, Lee J H, Kim H, et al. Bioinformatic analysis of proteomic data for iron, inflammation, and hypoxic pathways in restless legs syndrome. Sleep Med. 2020; 75:448-455. doi:10.1016/j.sleep.2020.09.002
Mondello S, Kobeissy F H, Mechref Y, et al. Searching for Novel Candidate Biomarkers of RLS in Blood by Proteomic Analysis. Nat Sci Sleep. 2021; 13:873-883. doi:10.2147/nss.S311801
Hanif U R, Leary E B, Schneider L D, et al. Estimation of Apnea-Hypopnea Index using Deep Learning on 3D Craniofacial Scans. IEEE J Biomed Health Inform. 2021; Pp. doi:10.1109/jbhi.2021.3078127
Leary E, Malunjkar S, Qadri S, et al. Deployment of the Alliance Sleep Questionnaire (ASQ) at the Stanford Sleep Disorders Clinic (SSDC) as Standard of Care. Sleep. 2013; 36 (Suppl. I): A413.
Moore Ht, Leary E, Lee S Y, et al. Design and validation of a periodic leg movement detector. PLOS One. 2014; 9 (12): e114565. doi:10.1371/journal.pone.0114565
Leary E B, Moore HEt, Schneider L D, Finn L A, Peppard P E, Mignot E. Periodic limb movements in sleep: Prevalence and associated sleepiness in the Wisconsin Sleep Cohort. Clin Neurophysiol. 2018; 129 (11): 2306-2314. doi:10.1016/j.clinph.2018.08.022
Ferri R, Fulda S, Allen R P, et al. World Association of Sleep Medicine (WASM) 2016 standards for recording and scoring leg movements in polysomnograms developed by a joint task force from the International and the European Restless Legs Syndrome Study Groups (IRLSSG and EURLSSG). Sleep Med. 2016; 26:86-95. doi:10.1016/j.sleep.2016.10.010
Allen R P, Picchietti D L, Garcia-Borreguero D, et al. Restless legs syndrome/Willis-Ekbom disease diagnostic criteria: updated International Restless Legs Syndrome Study Group (IRLSSG) consensus criteria—history, rationale, description, and significance. Sleep Med. 2014; 15 (8): 860-873. doi:10.1016/j.sleep.2014.03.025
Leary E, Malunjkar S, Ruoff C, Walsh J, Mignot E. Validation of the Alliance Sleep Questionnaire (ASQ) Restless Legs Syndrome Module Module in Sleep Disordered Patients. Sleep. 2015; 38.
Gold L, Ayers D, Bertino J, et al. Aptamer-based multiplexed proteomic technology for biomarker discovery. PLOS One. 2010; 5 (12): e15004. doi:10.1371/journal.pone.0015004
Kraemer S, Vaught J D, Bock C, et al. From SOMAmer-based biomarker discovery to diagnostic and clinical applications: a SOMAmer-based, streamlined multiplex proteomic assay. PLOS One. 2011; 6 (10): e26332. doi:10.1371/journal.pone.0026332
Manichaikul A, Mychaleckyj J C, Rich S S, Daly K, Sale M, Chen W M. Robust relationship inference in genome-wide association studies. Bioinformatics. 2010; 26 (22): 2867-2873. doi:10.1093/bioinformatics/btq559
Chang C C, Chow C C, Tellier L C, Vattikuti S, Purcell S M, Lee J J. Second-generation PLINK: rising to the challenge of larger and richer datasets. Gigascience. 2015; 4:7. doi:10.1186/s13742-015-0047-8
Purcell S, Neale B, Todd-Brown K, et al. PLINK: a tool set for whole-genome association and population-based linkage analyses. Am J Hum Genet. 2007; 81 (3): 559-575. doi:10.1086/519795
Seabold S, Perktold J. Statsmodels: Econometric and statistical modeling with python. In: proceedings from the Proceedings of the 9th Python in Science Conference; 2010.
Rijsman R M, de Weerd A W, Stam C J, Kerkhof G A, Rosman J B. Periodic limb movement disorder and restless legs syndrome in dialysis patients. Nephrology (Carlton). 2004; 9 (6): 353-361. doi:10.1111/j.1440-1797.2004.00330.x
Velayudham B, Thomas R G, Vasudevan C, Senthilkumar R. Serum cystatin C unmasks renal dysfunction in cirrhosis and performs better in estimation of glomerular filtration rate. Saudi Journal of Kidney Diseases and Transplantation. 2020; 31 (6): 1320.
Chen J, Bardes E E, Aronow B J, Jegga A G. ToppGene Suite for gene list enrichment analysis and candidate gene prioritization. Nucleic Acids Res. 2009; 37 (Web Server issue): W305-311. doi:10.1093/nar/gkp427
Pedregosa F, Varoquaux G, Gramfort A, et al. Scikit-learn: Machine learning in Python. the Journal of machine Learning research. 2011; 12:2825-2830.
Li J, Moore Ht, Lin L, et al. Association of low ferritin with PLM in the Wisconsin Sleep Cohort. Sleep Med. 2015; 16 (11): 1413-1418. doi:10.1016/j.sleep.2015.05.015
Wang T, Xu J, Xu Q, et al. Peripheral Iron Metabolism is Associated with Leg Movements on Polysomnography but Not with the Severity of Restless Legs Syndrome or Its Impact on Patients. Nat Sci Sleep. 2022; 14:1829-1842. doi:10.2147/nss.S378970
Chenini S, Delaby C, Rassu A L, et al. Hepcidin and ferritin levels in restless legs syndrome: a case-control study. Scientific reports. 2020; 10 (1): 11914. doi:10.1038/s41598-020-68851-0
Lemos Ados R, Ismael L A, Boato C C, Borges M T, Rondó P H. Hepcidin as a biochemical parameter for the assessment of iron deficiency anemia. Rev Assoc Med Bras (1992). 2010; 56 (5): 596-599. doi:10.1590/s0104-42302010000500024
Kumar S, Bhatia P, Jain R, Bharti B. Plasma Hepcidin Levels in Healthy Children: Review of Current Literature Highlights Limited Studies. J Pediatr Hematol Oncol. 2019; 41 (3): 238-242. doi:10.1097/mph.0000000000001216
Manolov V E, Atanasova B D, Velizarova M G, Vasilev V G, Tzatchev K N. Serum hepcidin levels in Bulgarian population. Clin Lab. 2014; 60 (12): 2001-2006. doi:10.7754/clin.lab.2014.140322
Sangkhae V, Nemeth E. Regulation of the Iron Homeostatic Hormone Hepcidin. Adv Nutr. 2017; 8 (1): 126-136. doi:10.3945/an.116.013961
Vogt A S, Arsiwala T, Mohsen M, Vogel M, Manolova V, Bachmann M F. On Iron Metabolism and Its Regulation. Int J Mol Sci. 2021; 22 (9). doi:10.3390/ijms22094591
Allen R P, Earley C J. The role of iron in restless legs syndrome. Mov Disord. 2007; 22 Suppl 18: S440-448. doi:10.1002/mds.21607
Trotti L M, Bhadriraju S, Becker L A. Iron for restless legs syndrome. Cochrane Database Syst Rev. 2012; 5 (5): Cd007834. doi:10.1002/14651858.CD007834.pub2.
Trotti L M, Becker L A. Iron for the treatment of restless legs syndrome. Cochrane Database Syst Rev. 2019; 1 (1): Cd007834. doi:10.1002/14651858.CD007834.pub3
Manconi M, Garcia-Borreguero D, Schormair B, et al. Restless legs syndrome. Nat Rev Dis Primers. 2021; 7 (1): 80. doi:10.1038/s41572-021-00311-z
Gossard T R, Trotti L M, Videnovic A, St Louis E K. Restless Legs Syndrome: Contemporary Diagnosis and Treatment. Neurotherapeutics. 2021; 18 (1): 140-155. doi:10.1007/s13311-021-01019-4
Bell S, Rigas A S, Magnusson M K, et al. A genome-wide meta-analysis yields 46 new loci associating with biomarkers of iron homeostasis. Commun Biol. 2021; 4 (1): 156. doi:10.1038/s42003-020-01575-z
Moksnes M R, Graham S E, Wu K H, et al. Genome-wide meta-analysis of iron status biomarkers and the effect of iron on all-cause mortality in HUNT. Commun Biol. 2022; 5 (1): 591. doi:10.1038/s42003-022-03529-z
Dowsett J, Didriksen M, Larsen M H, et al. No association between plasma hepcidin levels and restless legs syndrome-results from the Danish Blood Donor Study. Sleep Med. 2021; 88:68-73. doi:10.1016/j.sleep.2021.10.008
Dauvilliers Y, Chenini S, Vialaret J, et al. Association between serum hepcidin level and restless legs syndrome. Mov Disord. 2018; 33 (4): 618-627. doi:10.1002/mds.27287
Mizuno S, Mihara T, Miyaoka T, Inagaki T, Horiguchi J. CSF iron, ferritin and transferrin levels in restless legs syndrome. J Sleep Res. 2005; 14 (1): 43-47. doi:10.1111/j.1365-2869.2004.00403.x
Connor J R, Boyer P J, Menzies S L, et al. Neuropathological examination suggests impaired brain iron acquisition in restless legs syndrome. Neurology. 2003; 61 (3): 304-309. doi:10.1212/01.wnl.0000078887.16593.12
Barateau L, Chenini S, Lotierzo M, et al. CSF and serum ferritin levels in narcolepsy type 1 comorbid with restless legs syndrome. Ann Clin Transl Neurol. 2020; 7 (6): 924-931. doi:10.1002/acn3.51056
Beliveau V, Stefani A, Birkl C, et al. Revisiting brain iron deficiency in restless legs syndrome using magnetic resonance imaging. Neuroimage Clin. 2022; 34:103024. doi:10.1016/j.nicl.2022.103024
Hiraiwa M. Cathepsin A/protective protein: an unusual lysosomal multifunctional protein. Cell Mol Life Sci. 1999; 56 (11-12): 894-907. doi:10.1007/s000180050482
Krueger S, Bernhardt A, Kalinski T, et al. Induction of premalignant host responses by cathepsin x/z-deficiency in Helicobacter pylori-infected mice. PLOS One. 2013; 8 (7): e70242. doi:10.1371/journal.pone.0070242
Pišlar A H, Zidar N, Kikelj D, Kos J. Cathepsin X promotes 6-hydroxydopamine-induced apoptosis of PC12 and SH-SY5Y cells. Neuropharmacology. 2014; 82:121-131. doi:10.1016/j.neuropharm.2013.07.040
Weinstock L B, Walters A S, Paueksakon P. Restless legs syndrome—theoretical roles of inflammatory and immune mechanisms. Sleep Med Rev. 2012; 16 (4): 341-354. doi:10.1016/j.smrv.2011.09.003
Guo J, Pei L, Chen L, et al. Bidirectional association between irritable bowel syndrome and restless legs syndrome: a systematic review and meta-analysis. Sleep Med. 2021; 77:104-111. doi:10.1016/j.sleep.2020.12.002
Bellei E, Bergamini S, Monari E, et al. Evaluation of potential cardiovascular risk protein biomarkers in high severity restless legs syndrome. J Neural Transm (Vienna). 2019; 126 (10): 1313-1320. doi:10.1007/s00702-019-02051-7
Candia J, Cheung F, Kotliarov Y, et al. Assessment of Variability in the SOMAscan Assay. Scientific reports. 2017; 7 (1): 14248. doi:10.1038/s41598-017-14755-5
Kim C H, Tworoger S S, Stampfer M J, et al. Stability and reproducibility of proteomic profiles measured with an aptamer-based platform. Scientific reports. 2018; 8 (1): 8382. doi:10.1038/s41598-018-26640-w
Raffield L M, Dang H, Pratte K A, et al. Comparison of Proteomic Assessment Methods in Multiple Cohort Studies. Proteomics. 2020; 20 (12): e1900278. doi:10.1002/pmic.201900278

1. A method of treating restless leg syndrome (RLS) or periodic leg movement (PLM) in an individual, the method comprising:
administering to the individual an effective dose of an inhibitor of Sulfotransferase 1A3 (SULT1A3).

2. The method of claim 1, wherein the inhibitor is a small molecule drug.

3. The method of claim 1, wherein the inhibitor is tetrahydropterin.

4. The method of claim 1, wherein the individual has been diagnosed with RLS or PLM prior to treatment by a method comprising:
obtaining a blood sample from the individual;
analyzing the sample to quantitate levels of one or more biomarkers selected from ferritin light chain and heavy chain; LEAP1 (HAMP; Hepcidin); cathepsin A; megalin; sulfotransferase 1A3 (SULT1A3), and hepatocyte growth factor-regulated tyrosine kinase substrate (HRS), wherein altered levels of a biomarker relative to a normal control is indicative of RLS or PLM.

5. The method of claim 4, wherein the blood sample is plasma.

6. A method of determining or stratifying an individual for restless leg syndrome (RLS) or periodic leg movement (PLM), the method comprising:
obtaining a blood sample from the individual;
analyzing the sample to quantitate levels of one or more biomarkers selected from ferritin light chain and heavy chain; LEAP1 (HAMP; Hepcidin); cathepsin A; megalin; sulfotransferase 1A3 (SULT1A3), and hepatocyte growth factor-regulated tyrosine kinase substrate (HRS), wherein altered levels of a biomarker relative to a normal control is indicative of RLS or PLM.

7. The method of claim 4, wherein the biomarkers are ferritin, hepcidin and cathepsin A.

8. The method of claim 6, wherein decreased levels of ferritin and hepcidin, increased levels of cathepsin A, and the presence of RLS is diagnostic for PLM.

9. The method of claim 6, wherein increased levels of SULT1A3 and HRS relative to a normal control are indicative of PLM or RLS.

10. The method of claim 6, wherein the individual is treated in accordance with the determination.

11. The method of claim 10, wherein an individual diagnosed with PLM or RLS is treated with an effective dose of an inhibitor of SULT1A3.

12. The method of claim 10, wherein an individual diagnosed with PLM or RLS is treated with a dopamine agonist.

13. The method of claim 12, wherein the dopamine agonist is selected from pramipexole, ropinirole, and rotigotine.
